(19) United States
(12) Patent Application Publication
(10) Pub. No.: US 2009/0318817 A1
(43) Pub. Date: Dec. 24, 2009

(54) Pulse detection apparatus and method for manufacturing the same

(75) Inventors: Satoshi Sakurai, Shinagawa (JP); Ayumu Akabane, Shinagawa (JP); Takashi Arita, Shinagawa (JP)

Correspondence Address: STAAS & HALSEY LLP, SUITE 700, 1201 NEW YORK AVENUE, N.W., WASHINGTON, DC, 20005, US

(73) Assignee: FUJITSU COMPONENT LIMITED, Tokyo (JP)

(21) Appl. No.: 12/457,699

(22) Filed: Jun. 18, 2009

(30) Foreign Application Priority Data
Jun. 20, 2008 (JP) 2008-162394

(51) Int. Cl. A61B 5/02

(52) U.S. Cl. 600/490

(57) ABSTRACT

A pulse detection apparatus including: a support member that supports a pulse sensor so as to touch a human body with the pulse sensor; a cable that is electrically connected to the pulse sensor and mechanically connected to the support member; a first restriction member that is provided on the cable, and restricts the movement of the cable in a lateral direction thereof by fitting in a first concave portion provided on the support member; and a second restriction member that is provided on the cable, and restricts the movement of the cable in an extension direction thereof by fitting in a second concave portion provided on the support member.

### BACKGROUND OF THE INVENTION

1. Field of the Invention

The present invention relates to a pulse detection apparatus, and a method for manufacturing the pulse detection apparatus, and more particularly to a pulse detection apparatus in which a cable is connected to a support member supporting a pulse sensor, and a method for manufacturing the pulse detection apparatus.

2. Description of the Related Art

A pulse detection apparatus used for acquiring pulse information relating to a user's pulse acquires the pulse information by putting a pulse sensor on an auricle, for example. The acquired pulse information is processed with a portable device, and stored into the portable device. Japanese Patent Application Publication No. 2005-185725 discloses the pulse detection apparatus.

A support member supporting the pulse sensor makes the pulse sensor touch a part of the human body. Thereby, the pulse sensor acquires information relating to the pulse. However, when a user puts the pulse sensor on the user's body and exercises, the cable for outputting a signal from the pulse sensor can be easily come away from the support member. The cable is horizontally moved to the support member, so that it is easy to damage the cable in a part where the cable is connected with the support member.

### SUMMARY OF THE INVENTION

It is an object of the present invention to provide a pulse detection apparatus and a method for manufacturing the pulse detection apparatus that can avoid damaging a cable.

According to an aspect of the present invention, there is provided a pulse detection apparatus including: a support member that supports a pulse sensor so as to touch a human body with the pulse sensor; a cable that is electrically connected to the pulse sensor and mechanically connected to the support member; a first restriction member that is provided on the cable, and restricts the movement of the cable in a lateral direction thereof by fitting in a first concave portion provided on the support member; and a second restriction member that is provided on the cable, and restricts the movement of the cable in an extension direction thereof by fitting in a second concave portion provided on the support member.

With the above arrangement, the movement of the cable in the lateral direction is restricted by the first restriction member, and hence it is possible to avoid the disconnection of the cable by the movement of the cable in the lateral direction. In addition, the movement of the cable in the extension direction is restricted by the second restriction member, and hence it can be avoided that the cable comes away from the support member.

### BRIEF DESCRIPTION OF THE DRAWINGS

Preferred embodiments of the present invention will be described in detail with reference to the following drawings, wherein:

FIG. 1 is a block diagram showing a pulse detection apparatus according to a first embodiment of the present invention;

FIG. 2 is a diagram showing the appearance of the pulse detection apparatus according to the first embodiment of the present invention;

FIGS. 3A and 3B are exploded perspective views showing a support member supporting a pulse sensor;

FIG. 4 is a diagram showing a state where the support member nips a part of a human body;

FIG. 5A is a side view showing the support member;

FIG. 5B is a rear view showing the support member;

FIG. 6A is a rear view showing a lower part of the support member;

FIG. 6B is a top view showing a part of the lower part of the support member;

FIG. 7 is a top view showing a first restriction member and a second restriction member;

FIGS. 8A to 8C are diagrams showing processes in which a cable is connected to the support member;

FIG. 9 is a rear perspective view showing the lower part according to a second embodiment of the present invention; and

FIGS. 10A to 10C are diagrams showing processes in which the cable is connected to the support member.

### DESCRIPTION OF THE PREFERRED EMBODIMENTS

A description will now be given, with reference to the accompanying drawings, of embodiments of the present invention.

### First Embodiment

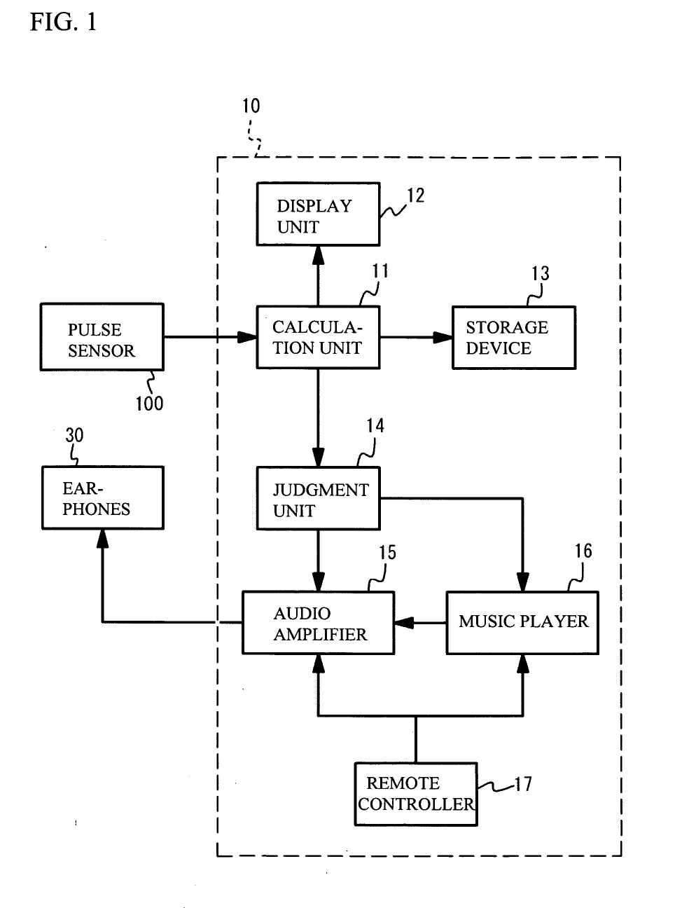

FIG. 1 is a block diagram showing a pulse detection apparatus according to a first embodiment of the present invention. The pulse detection apparatus includes a portable device 10, a pulse sensor 100, and earphones 30. Pulse waves detected by the pulse sensor 100 are output to a calculation unit 11. The calculation unit 11 is composed of a CPU, for example, calculates pulse information relating to pulses, and outputs the result of the calculation to a display unit 12. The display unit 12 is composed of a liquid crystal display unit, for example, and displays the result of the calculation. The result of the calculation indicates the number of pulses calculated from the pulse waves, for example. A storage device 13 is composed of a nonvolatile memory, for example, and stores pulse biological information including the pulse information, the result of the calculation, or the like. The calculation unit 11 outputs the result of the calculation to a judgment unit 14.

The judgment unit 14 judges a user's living body situation (e.g. a relaxation state and a tension state) based on the result of the calculation. The judgment unit 14 controls an audio amplifier 15 and a music player 16 based on the result of the judgment. The music player 16 may be composed of a radio receiver, a portable game player, a portable phone, or the like, other than the exclusive music player. The audio amplifier 15 amplifies sound output from the music player 16, and outputs the amplified sound to the earphones 30. A remote controller 17 is used to perform the operation of a volume of the audio amplifier 15 and the operation (e.g. selection of music) of the music player 16. When the number of pulses of the user is settled down, for example, the judgment unit 14 judges that the user's living body situation is in the relaxation state. Then, the judgment unit 14 causes the music player 16 to select a music with a slow tempo, and causes the audio amplifier 15 to lower the volume thereof.

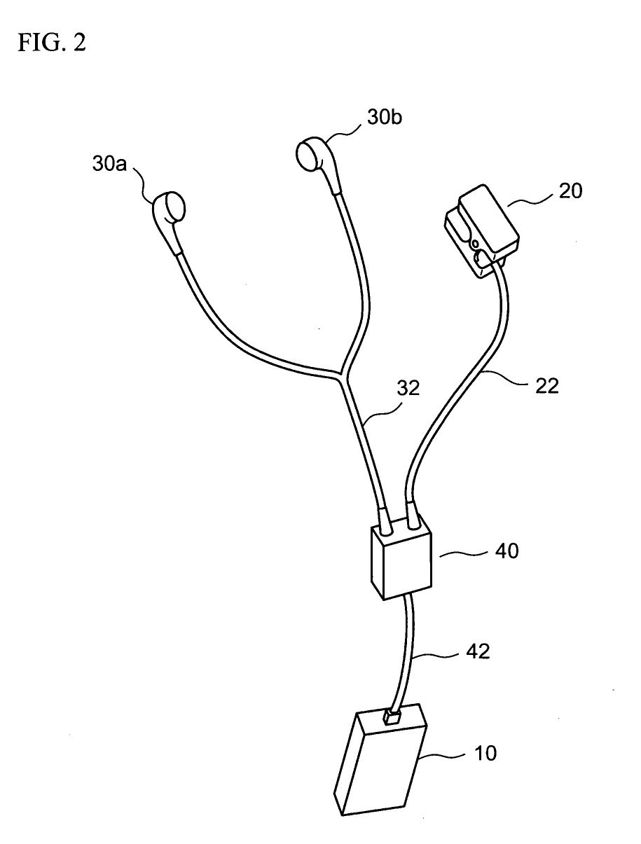

FIG. 2 is a diagram showing the appearance of the pulse detection apparatus according to the first embodiment of the present invention. The pulse sensor 100 is supported by a support member 20. The support member 20 and the portable device 10 are connected to each other via cables 22 and 42. An earphone connection unit 40 is provided between the cables 42 and 22. Earphones 30a and 30b are connected to the portable device 10 via a cable 32, the earphone connection unit 40, and the cable 42. The portable device 10 is attached to a belt for the waist and a pocket of a jacket, for example. The earphones 30a and 30b are inserted into external auditory canals. The support member 20 is put on an earlobe, for example.

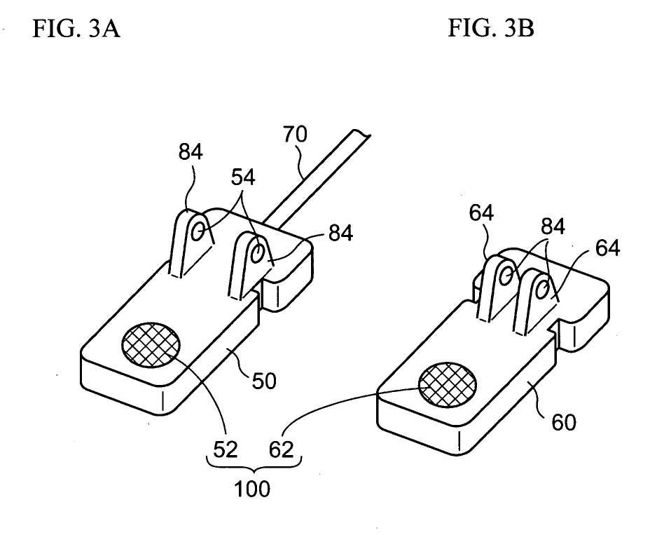

FIGS. 3A and 3B are exploded perspective views showing the support member 20 supporting the pulse sensor 100. The support member 20 includes a lower part 50 and an upper part 60. Referring to FIG. 3A, an upper section 52 of the pulse sensor 100 is provided at an area which touches the earlobe and is provided on a upper face of the lower part 50. The upper section 52 of the pulse sensor 100 has a light receiving element such as a photodiode, windows guiding infrared rays, and so on. A cable 70 corresponding to the cable 22 is mechanically connected to a rear face of the lower part 50. Protrusions 54 are provided on the upper face of the lower part 50, and a through-hole 84 is provided on each of the protrusions 54. Referring to FIG. 3B, a lower section 62 of the pulse sensor 100 is provided at an area which touches the earlobe and is provided on a lower face of the upper part 60. The lower section 62 of the pulse sensor 100 has a light receiving element such as a photodiode, windows guiding infrared rays, and so on. Protrusions 64 are provided on the lower face of the upper part 60, and the through-hole 84 is provided on each of the protrusions 64. The lower part 50 and the upper part 60 are formed by a resin such as an ABS (Acrylonitrile Butadiene Styerene) resin.

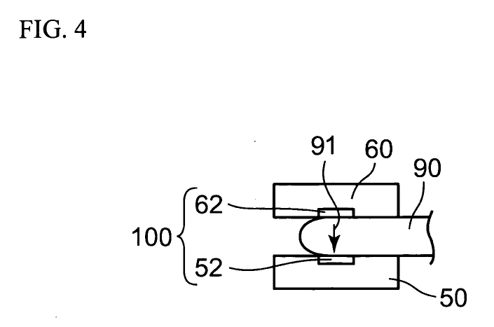

FIG. 4 is a schematic diagram showing a state where the lower part 50 and the upper part 60 nip a part of a human body such as an earlobe 90. Referring to FIG. 4, an infrared ray 91 emitted from the lower section 62 of the pulse sensor 100 to the upper section 52 thereof penetrates the earlobe 90. The penetration amount of the infrared ray changes by the pulse. Therefore, the pulse wave can be detected by detecting the penetration amount of the infrared ray. Not only a sensor detecting the pulse wave by the penetration of the infrared ray but also a sensor detecting the pulse wave by the change of capacitance can be used as the pulse sensor 100.

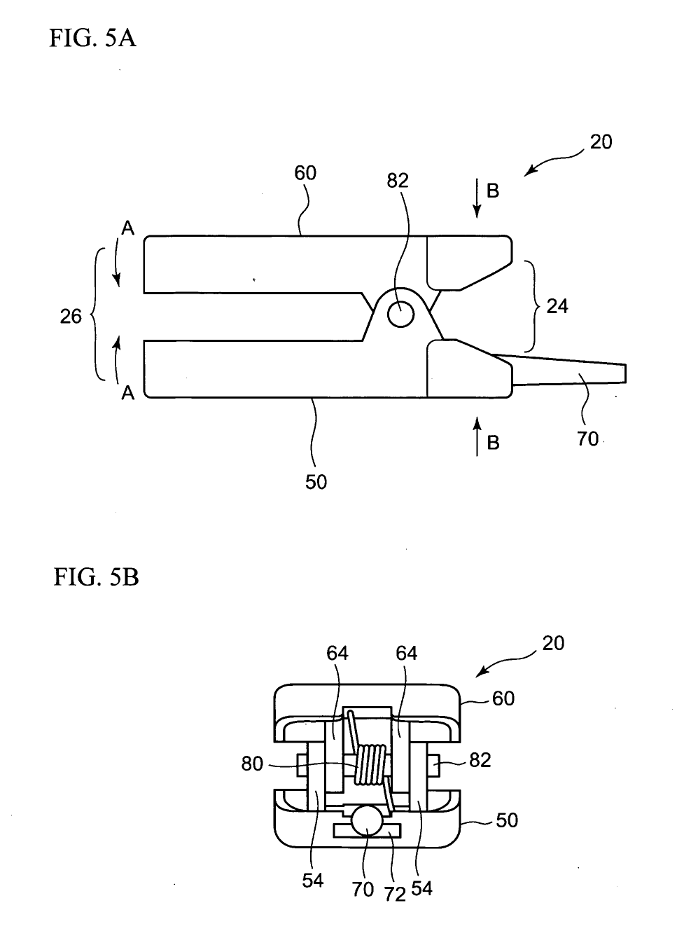

FIG. 5A is a side view showing the support member 20 of the pulse sensor 100, and FIG. 5B is a rear view showing the support member 20. Referring to FIGS. 5A and 5B, a shaft 82 passes through through-holes 84, so that the lower part 50 and the upper part 60 are integrated with each other. The lower part 50 and the upper part 60 are capable of pivotally moving about the shaft 82. A nip section 26 that nips the part of the human body is provided at a front end of the support member 20. A hold section 24 is provided at a rear side (i.e., a side of the cable 70) of the support member 20. The nip section 26 is biased to nip the part of the human body (see arrows “A” in FIG. 5A). The nip section 26 can be opened by nipping the hold section 24 by the finger (see arrows “B” in FIG. 5A). For example, after the hold section 24 is nipped by the finger as shown by the arrows “B”, and the nip section 26 is placed to surround the earlobe, the hold section 24 is released. Thereby, the nip section 26 can nip the earlobe as shown in FIG. 4.

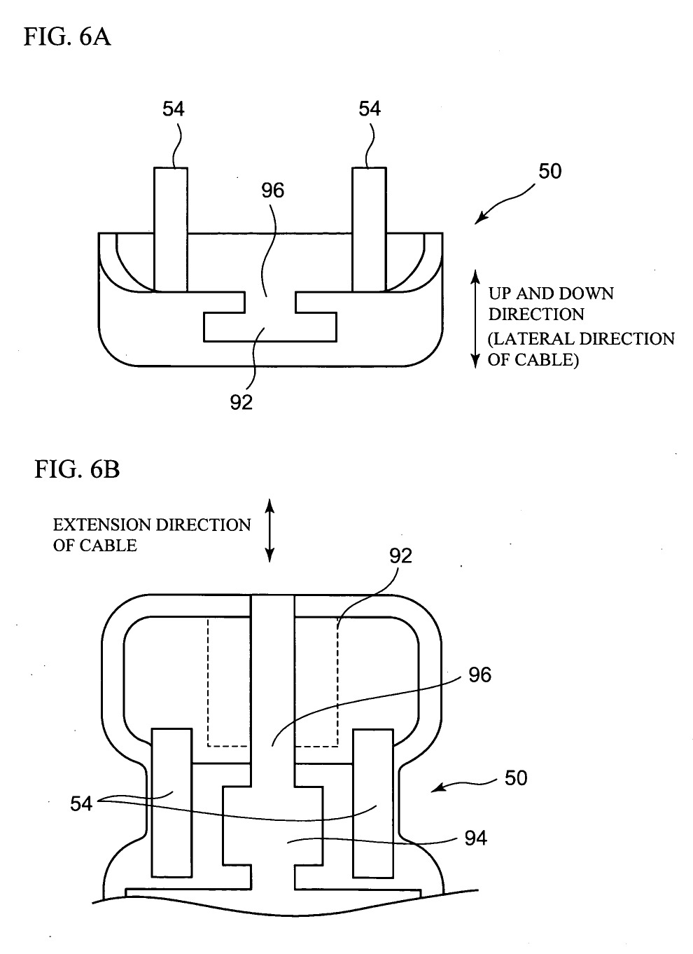

FIG. 6A is a rear view showing the lower part 50 of the support member. Referring to FIG. 6A, a first concave portion 92 having a depth in an extension direction of the cable 70 is provided on the rear face of the lower part 50. A guide groove 96 for the cable 70 is provided on an upper side of the first concave portion 92. The guide groove 96 has an opening in a direction where a second concave portion 94 opens. FIG. 6B is a top view showing a part of the lower part 50. Referring to FIG. 6B, the second concave portion 94 having a depth in a down direction (i.e., a lateral direction of the cable 70 or a depth direction of FIG. 6B) is provided on the upper face of the lower part 50. The lateral direction of the cable 70 indicates a up and down direction or a right and left direction perpendicular to the extension direction of the cable 70.

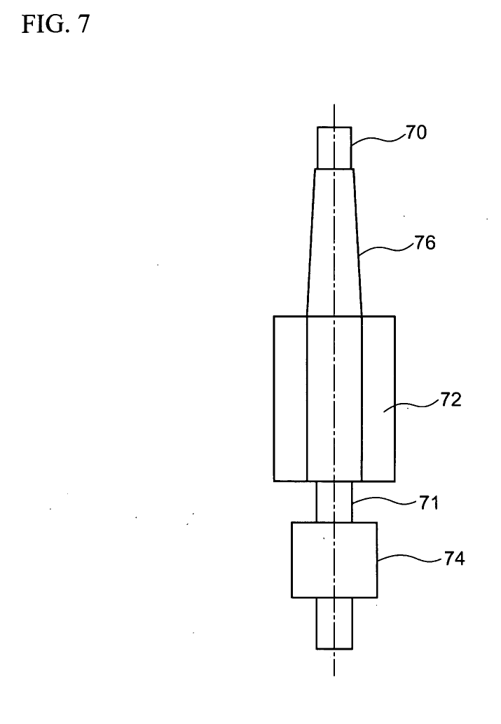

FIG. 7 show a top view of the cable 70. Referring to FIG. 7, a skirt member 76, a first restriction member 72 and a second restriction member 74 are arranged in the extension direction of the cable 70 and formed on the cable 70 so as to nip a part 71 of the cable 70. The skirt member 76 has a skirt form that increasingly thickens in a direction of the support member 20, and a function to restrict bending the cable 70 at the root of the support member 20. The first restriction member 72 fits in the first concave portion 92, to thereby restrict the movement of the cable 70 in the up and down direction or the right and left direction (i.e., the lateral direction of the cable 70; see FIG. 6A). The second restriction member 74 fits in the second concave portion 94, and hence it is restricted that the second restriction member 74 comes away from the support member 20 in the extension direction of the cable 70 (see FIG. 6B). Each of the first restriction member 72 and the second restriction member 74 has a wing shape extended in the right and left direction perpendicular to the extension direction of the cable 70, and is composed of a rubber or the resin such as the ABS resin.

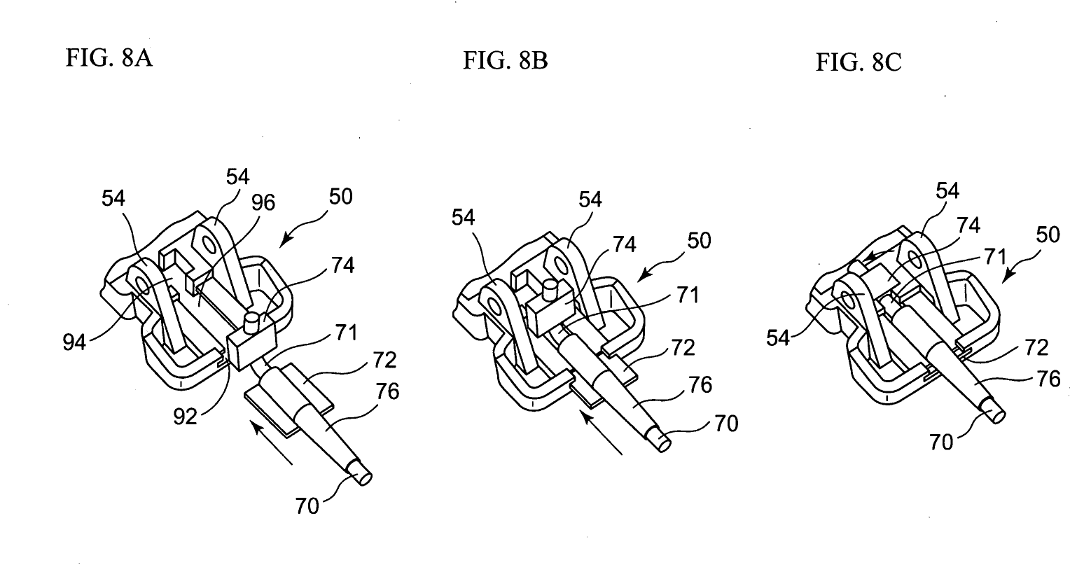

FIGS. 8A to 8C are diagrams showing processes in which the cable 70 is mechanically connected to the support member 20. Referring to FIGS. 8A and 8B, the first restriction member 72 are inserted into the first concave portion 92 along the extension direction of the cable 70 as shown by the arrow. At this time, the second restriction member 74 is bent upward, and the part 71 of the cable 70 arranged between the first restriction member 72 and the second restriction member 74 is guided to the guide groove 96. Thereby, the first restriction member 72 fits in the first concave portion 92. Referring to FIG. 8C, after the first restriction member 72 is inserted into the first concave portion 92, the second restriction member 74 is inserted into the second concave portion 94 along a down direction (i.e., the lateral direction of the cable 70) as shown by the arrow. Thereby, the second restriction member 74 fits in the second concave portion 94. A front end of the cable 70 is arranged in the lower part 50 and connected to a substrate (not show) with a solder, or the like. Accordingly, the cable 70 is electrically connected to the pulse sensor 100 and mechanically connected to the support member 20.

According to the first embodiment, the movement of the cable 70 in the lateral direction is restricted by the first restriction member 72, and hence it is possible to avoid the disconnection of the cable 70 by the movement of the cable 70 in the lateral direction. The movement of the cable 70 in the extension direction is restricted by the second restriction member 74, and hence it can be avoided that the cable 70 comes away from the support member 20. Thus, the first restriction member 72 fits in the first concave portion 92 and the second restriction member 74 fits in the second concave portion 94, so that the movement of the cable 70 in the lateral direction and the extension direction can be restricted. As a result, the damage of the cable 70 can be avoided by an easy manufacturing process.

Also, as shown in FIG. 8C, the first restriction member 72 is inserted into the first concave portion 92 along the extension direction of the cable 70, so that the movement of the cable 70 in the up and down direction, and the right and left direction is restricted. The second restriction member 74 is inserted into the second concave portion 94 along the lateral direction of the cable 70, so that the movement of the cable 70 in the extension direction is restricted. Thereby, it can be avoided that the cable 70 comes away from the support member 20 in the extension direction. The first restriction member 72 is inserted into the first concave portion 92 and the second restriction member 74 is inserted into the second concave portion 94, and hence the pulse detection apparatus which avoids damaging the cable can be easily manufactured.

In addition, the guide groove 96 is provided on the same face as an opening section of the second concave portion 94 as shown in FIG. 6B, and the part 71 of the cable 70 is guided by the guide groove 96 as shown in FIG. 8B. With the configuration, the part 71 of the cable 70 is guided by the guide groove 96, so that the pulse detection apparatus can be easily manufactured.

### Second Embodiment

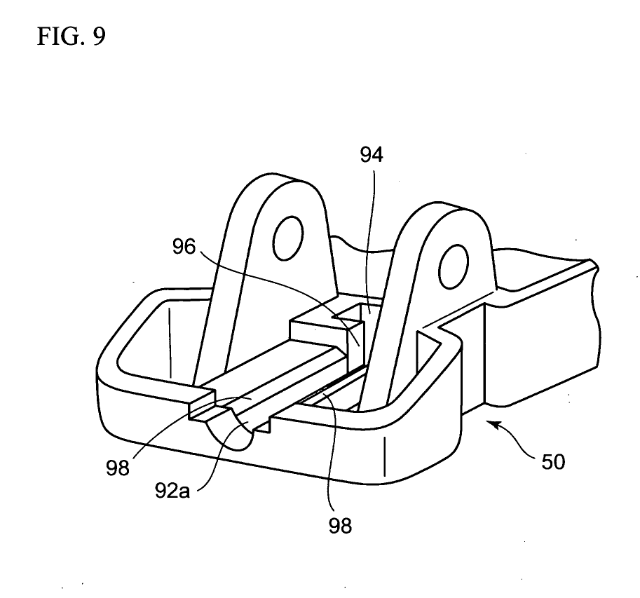

FIG. 9 is a rear perspective view showing the lower part of the support member according to a second embodiment.

Referring to FIG. 9, the lower part 50 has a third restriction member 98 which is arranged above a first concave portion 92a and protrudes from right and left directions. The third restriction member 98 restricts that the first restriction member 72 comes away from the support member 20 in an insertion direction. A second concave portion 94a indicates a concave portion having a depth in the down direction (i.e., the lateral direction of the cable 70). Other configurations are identical with those in the first embodiment, and description thereof is omitted.

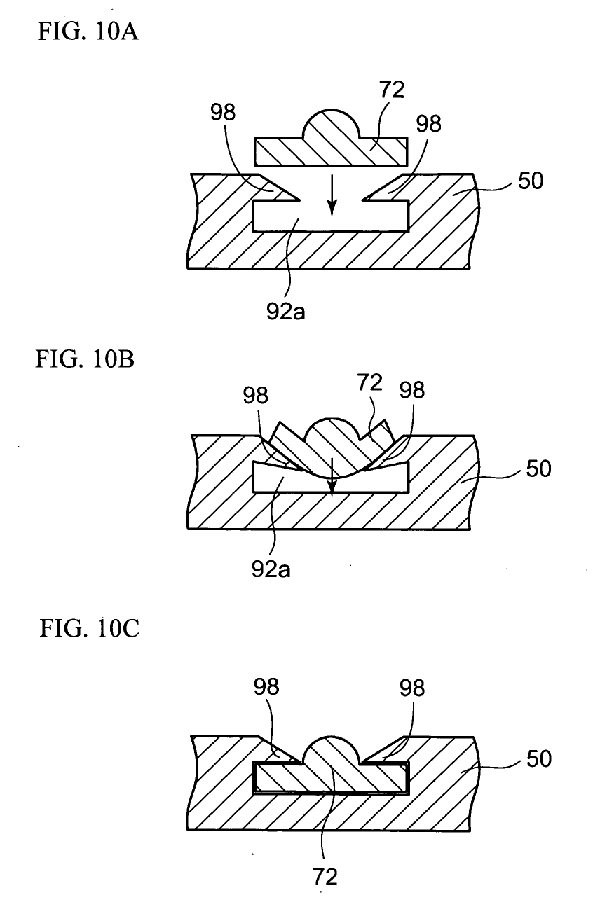

FIGS. 10A to 10C are diagrams showing processes in which the cable 70 is connected to the support member 20, and are schematic cross-sectional views showing cutting surfaces in the first concave portion 92a of the lower part 50, as viewed from the rear. Referring to FIG. 10A, the third restriction member 98 protrudes from right and left directions, and the tip thereof has a thin taper. The first restriction member 72 is push down toward the first concave portion 92a (i.e., in the lateral direction of the cable 70; the arrow direction in FIG. 10A). Referring to FIG. 10B, the third restriction member 98 and the first restriction member 72 are transformed. Referring to FIG. 10C, the first restriction member 72 gets over the third restriction member 98, and is inserted into the first concave portion 92a. Thereby, the first restriction member 72 fits in the first concave portion 92a.

According to the second embodiment, the third restriction member 98 is arranged on an upper face of the first restriction member 72, and hence the first restriction member 72 restricts coming away from the support member 20 in the lateral direction of the cable 70 (i.e., the insertion direction of the cable 70). In the second embodiment, the insertion direction of the first restriction member 72 is the same as that of the second restriction member 74, and hence the first restriction member 72 and the second restriction member 74 can easily fit in the first concave portion 92a and the second concave portion 94, respectively. In the second embodiment, the first restriction member 72 and the third restriction member 98 are formed with elastic and transformable materials, as shown in FIG. 10B. On the other hand, in the first embodiment, the first restriction member 72 can be formed with relatively hard materials.

Although the first and second embodiments indicate examples of the pulse detection apparatus in which the support member 20 nips the part of the human body and the pulse sensor touch part of the human body, the support member 20 may cause the pulse sensor to touch the part of the human body by other ways.

It should be noted that the present invention is not limited to the embodiments, and various modifications may be made to them without departing from the scope of the invention.

The Present application is based on Japanese Patent Application No. 2008-162394 filed Jun. 20, 2008, the entire disclosure of which is hereby incorporated by reference.

What is claimed is:

1. A pulse detection apparatus comprising:
a support member that supports a pulse sensor so as to touch a human body with the pulse sensor;
a cable that is electrically connected to the pulse sensor and mechanically connected to the support member;
a first restriction member that is provided on the cable, and restricts the movement of the cable in a lateral direction thereof by fitting in a first concave portion provided on the support member; and
a second restriction member that is provided on the cable, and restricts the movement of the cable in an extension direction thereof by fitting in a second concave portion provided on the support member.

2. The pulse detection apparatus as claimed in claim 1, wherein the first concave portion has a depth in the extension direction of the cable, and the second concave portion has a depth in the lateral direction of the cable.

3. The pulse detection apparatus as claimed in claim 2, wherein the first restriction member and the second restriction member are formed in the extension direction of the cable so as to nip a part of the cable, and a guide groove is provided on the first concave portion, the guide groove including an opening in a direction where the second groove portion opens, and guiding the part of the cable provided between the first restriction member and the second restriction member.

4. The pulse detection apparatus as claimed in claim 1, wherein the first concave portion has a depth in the lateral direction of the cable and includes a third restriction member which restricts that the first restriction member comes away from the support member in the lateral direction of the cable, and the second concave portion has a depth in the lateral direction of the cable.

5. The pulse detection apparatus as claimed in claim 1, wherein the support member causes the pulse sensor to touch the human body by nipping a part of the human body.

6. A method for manufacturing a pulse detection apparatus, comprising:
a first step of fitting a first restriction member provided on a cable in a first concave portion provided on a support member, the first restriction member restricting the movement of the cable in a lateral direction thereof, the support member supporting a pulse sensor so as to touch a human body with the pulse sensor, and the cable being electrically connected to the pulse sensor; and
a second step of fitting a second restriction member provided on the cable in a second concave portion provided on the support member, the second restriction member restricting the movement of the cable in an extension direction thereof.

7. The method for manufacturing the pulse detection apparatus as claimed in claim 6, wherein the first step includes a third step of inserting the first restriction member into the first concave portion along the extension direction of the cable, and the second step includes a fourth step of inserting the second restriction member into the second concave portion along the lateral direction of the cable.

8. The method for manufacturing the pulse detection apparatus as claimed in claim 7, wherein the first restriction member and the second restriction member are formed in the extension direction of the cable so as to nip a part of the cable, and the first step includes a fifth step of inserting the first restriction member into the first concave portion so that the part of the cable is guided by a guide groove including an opening in a direction where the second groove portion opens.

9. The method for manufacturing the pulse detection apparatus as claimed in claim 6, wherein the first step includes a sixth step of inserting the first restriction member into the first concave portion along the lateral direction of the cable, the first concave portion including a third restriction member which restricts that the first restriction member comes away from the support member in an insertion direction of the cable, and the second step includes a seventh step of inserting the second restriction member into the second concave portion along the lateral direction of the cable.
